(19) United States
(12) Patent Application Publication
(10) Pub. No.: US 2026/0128187 A1
(43) Pub. Date: May 7, 2026

(54) SYSTEM AND METHOD FOR POSITIONING RADIATION SHIELD

(71) Applicant: KONINKLIJKE PHILIPS N.V., EINDHOVEN (NL)

(72) Inventors: Leili SALEHI, WALTHAM, MA (US); Ayushi SINHA, CLEVELAND HEIGHTS, OH (US); Javad FOTOUHI, CAMBRIDGE, MA (US); Ramon Quido ERKAMP, SWAMPSCOTT, MA (US); Vipul Shrihari PAI RAIKAR, SOMERVILLE, MA (US); Sean Joseph KYNE, BROOKLINE, MA (US)

(21) Appl. No.: 19/117,456

(22) PCT Filed: Oct. 7, 2023
(86) PCT No.: PCT/EP2023/077805

§ 371 Date: Apr. 1, 2025

Related U.S. Application Data
(60) Provisional application No. 63/415,037, filed on Oct. 11, 2022.

(51) Int. Cl. G21F 3/00; A61B 6/00; A61B 6/10

(52) U.S. Cl. CPC G21F 3/00; A61B 6/107; A61B 6/542; A61B 6/547

(57) ABSTRACT

A system and method are provided for reducing radiation exposure in a procedure room using a radiation shield. The method determines first position data indicating a position of a clinician in the procedure room and determines second position data indicating a position of an imaging source of an imaging system in the procedure room. The method further applies a radiation model to estimate a radiation pattern of radiation emitted by the imaging source based on the first position data and the second position data. The method further applies a shield positioning model to predict an optimal position of the radiation shield that minimizes exposure of the at least one clinician to the emitted radiation based on the estimated radiation pattern, the first position data, and the second position data.

### BACKGROUND

Repeated exposure to high amounts of ionizing radiation may lead to health issues, such as erythema, hair loss, dermal atrophy, fibrosis, desquamation, dermal necrosis, cataracts, decrease in red blood cell production and infertility. For example, medical imaging that emits radiation (e.g., X-ray imaging) is needed to provide real-time and near real-time images during certain interventional procedures performed within a procedure room. Therefore, radiation exposure is a problem for many medical personnel, including physicians, radiologists, interventionists and staff, as well as for patients, located within the procedure room during repeated procedures involving the emission of radiation. For example, between 2012 and 2015, nine cases of left-sided brain/head-and-neck tumors in interventional cardiologists had been reported, according to a Cleveland Clinic report from 2015, entitled “Radiation a Danger to Patients and Physicians Alike” (https://consultqd.clevelandclinic.org/). Interventionalists may also receive increased doses of radiation to their hands during several procedures. Even low amounts of radiation exposure may damage the genetic material in reproductive cells and increase chromosomal abnormalities. Radiation exposure may also alter DNA over time, as studies have shown increases in chromosomal abnormalities in medical personnel who are interventionalists, compared with those who are non-interventionalists.

Long term presence of the medical personnel procedure rooms using X-ray imaging systems, for example, may cause some health issues caused by ionizing radiation. The amount of the radiation dose emitted towards the medical personnel depends on C-arm orientation and location of the radiation source, patient size and position, and locations of medical personnel and patient relative to the C-arm/radiation source and the operating table. Protective shields and lead jackets may reduce the received doses of radiation, however they have limitations and drawbacks that contribute to the dissatisfaction of the medical personnel. Indeed, the limited size of the protective shields above the operating table and sometimes its improper position and orientation may increase the amount of the radiation received by the medical personnel. In addition, protective lead jackets are cumbersome and heavy, and may cause musculoskeletal problems after long-term usage.

Protective shields may be provided in attempts to attenuate the radiation exposure. However, sometimes large amounts of radiation may still be received by the medical personnel due to factors such as inappropriate sizes, locations and/or orientations of the protective shields. Using more than one protective shield may increase protection, but it also contributes to room clutter and distraction.

Manual repositioning of protective shields is time consuming and distracting, and requires caution and attention with regard to constantly estimating the best position and orientation based on changing C-arm positions. Therefore, manual repositioning typically does not provide optimal positions of the protective shields during procedures in response to movements of the C-arm and/or the medical personnel.

Accordingly, there is a clinical need for reducing doses of radiation exposure of medical personnel by automatically repositioning the radiation protection shields based on the number and location of medical professionals standing near the patient on an operating table, objects near the operating table and the angle of the C-arm.

### SUMMARY

According to representative embodiments, a system is provided for reducing exposure to X-ray radiation of at least one clinician in a procedure room. The system includes a controller comprising a processor and memory, the processor configured to: receive first position data indicating a position of at least one clinician in a procedure room; receive second position data indicating a position of an imaging source of an imaging device configured to provide image data of a patient in the procedure room; estimate a radiation pattern of radiation emitted by the imaging source based on the first position data and the second position data; and predict an optimal position of a radiation shield in the procedure room that minimizes exposure of the at least one clinician to the emitted radiation based on the estimated radiation pattern, the first position data, and the second position data. The system may also include the radiation shield formed of radiation shielding material; and at least one sensor configured to provide the first position data indicating a position of the at least one clinician in the procedure room and the second position data indicating a position of an X-ray source of an X-ray imaging device configured to provide image data of a patient in the procedure room.

According to other representative embodiments, a method is provided for reducing exposure of at least one clinician to X-ray radiation from an X-ray source in a procedure room using a radiation shield. The method includes: determining first position data indicating a position of at least one clinician in a procedure room; determining second position data indicating a position of an imaging source of an imaging device configured to provide image data of a patient in the procedure room; estimating a radiation pattern of radiation emitted by the imaging source based on the first position data and the second position data; and predicting an optimal position of a radiation shield in the procedure room that minimizes exposure of the at least one clinician to the emitted radiation based on the estimated radiation pattern, the first position data, and the second position data.

According to other representative embodiments, a non-transitory computer readable medium is provided for storing instructions for reducing exposure of at least one clinician to X-ray radiation from an X-ray source in a procedure room using a radiation shield. When executed by one or more processors, the instructions cause the processor to: determine first position data indicating a position of at least one clinician in a procedure room; determine second position data indicating a position of an imaging source of an imaging device configured to provide image data of a patient in the procedure room; estimate a radiation pattern of radiation emitted by the imaging source based on the first position data and the second position data; and predict an optimal position of a radiation shield in the procedure room that minimizes exposure of the at least one clinician to the emitted radiation based on the estimated radiation pattern, the first position data, and the second position data.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a simplified block diagram of a system for reducing exposure of at least one clinician to X-ray radiation from an X-ray source in a procedure room, according to a representative embodiment.

FIG. 2A is a schematic view of radiation shield placement minimizing radiation exposure to the clinician, according to a representative embodiment.

FIG. 2B is a schematic view of radiation shield placement minimizing radiation exposure to the clinician, according to a representative embodiment.

FIG. 3 is a flow diagram of a method for reducing exposure of at least one clinician to X-ray radiation from an X-ray source in a procedure room using a radiation shield, according to a representative embodiment.

FIG. 4 is a flow diagram of a method for training a shield positioning model for reducing exposure of the at least one clinician to X-ray radiation from the X-ray source, according to a representative embodiment.

FIG. 5 is a diagram of a radiation shield system including a perspective view of a configurable radiation shield, according to a representative embodiment.

FIG. 6 is a perspective view of a connector movably connecting the first and second sections of the radiation shield for rotational movement, according to a representative embodiment.

FIG. 7A is a perspective view of a connector movably connecting the first and second sections of the radiation shield for translational movement, according to a representative embodiment.

FIG. 7B is a perspective view of a spool in the connector operable to provide translational movement of the first and second sections of the radiation shield, according to a representative embodiment.

FIG. 8 is a perspective view of a connector movably connecting the upper and lower panels in the third section of the radiation shield for vertical movement, according to a representative embodiment.

FIG. 9 is a perspective view of a connector movably connecting the upper and lower panels in the first section of the radiation shield for vertical movement, according to a representative embodiment.

### DETAILED DESCRIPTION

In the following detailed description, for purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only, and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only, and is not intended to be limiting. As used in the specification and appended claims, the singular forms of terms “a,” “an” and “the” are intended to include both singular and plural forms, unless the context clearly dictates otherwise. Additionally, the terms “comprises,” and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In view of the foregoing, the present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

Generally, the various embodiments provide systems and methods for autonomous manipulation of one or more radiation shields for protection of one or more clinicians in a procedure room from radiation during operation of an X-ray imaging system. In some embodiments, machine-learning algorithms use the position and/or tracking information from an X-ray source, which may be mounted on a C-arm, and one or more clinicians and the patient in the procedure room.

The system may be a stand-alone solution provided with its own radiation shield(s), or may be connected to radiation shield(s) already present in the procedure room and/or may be unified with an X-ray imaging system to directly receive the information about C-arm angulation for both radiation exposure reduction and collision prevention.

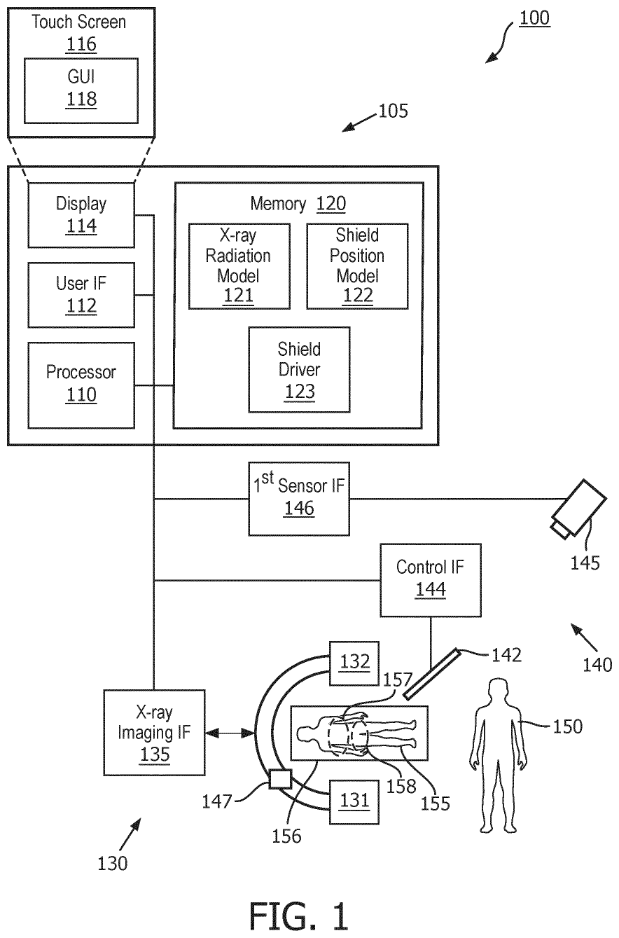

FIG. 1 is a simplified block diagram of a system for reducing exposure of at least one clinician to X-ray radiation from an X-ray source in a procedure room, according to a representative embodiment.

Referring to FIG. 1, system 100 includes a control unit (controller) 105, an imaging system (e.g., X-ray imaging system) 130, and a shield placement system 140. The control unit 105 is configured to implement and/or manage the processes described herein. The control unit 105 includes one or more processors indicated by processor 110, one or more memories indicated by memory 120, a user interface (IF) 112, and a display 114. The memory 120 stores instructions executable by the processor 110. When executed, the instructions cause the processor 110 to implement one or more processes for determining and reducing exposure to radiation of the at least one clinician, indicated by clinician 150, through manipulation of a radiation shield 142 of the shield placement system 140, as well as to control performance of the X-ray imaging system 130, as discussed below. As used herein, “clinician” refers to any personnel in the procedure room, such as an interventionalist, a radiology technician, an anesthesiologist, and a nurse, for example, each of whom may be exposed to radiation while performing a procedure on a patient 155. The procedure may be an interventional procedure, such as an interventional endovascular or endobronchial procedure (e.g., heart catheterization and transcatheter aortic valve replacement (TAVR)), for example. For purposes of illustration, the memory 120 is shown to include software modules, each of which includes the instructions corresponding to an associated capability of the control unit 105, as discussed below.

The processor 110 is representative of one or more processing devices, and may be implemented by a general purpose computer, a central processing unit (CPU), a computer processor, digital signal processor (DSP), a graphics processing unit (GPU), a microprocessor, a microcontroller, a state machine, programmable logic device, field programmable gate arrays (FPGAs), application specific integrated circuits (ASICs), or combinations thereof, using any combination of hardware, software, firmware, hard-wired logic circuits, or combinations thereof. Any processing device or processor herein may include multiple processors, parallel processors, or both. Multiple processors may be included in, or coupled to, a single device or multiple devices. The term “processor” as used herein encompasses an electronic component able to execute a program or machine executable instruction. A processor may also refer to a collection of processors within a single computer system or distributed among multiple computer systems, such as in a cloud-based or other multi-site application. Programs have software instructions performed by one or multiple processors that may be within the same computing device or which may be distributed across multiple computing devices.

The memory 120 may include main memory and/or static memory, where such memories may communicate with each other and the processor 110 via one or more buses. The memory 120 may be implemented by any number, type and combination of random access memory (RAM) and read-only memory (ROM), for example, and may store various types of information, such as software algorithms, artificial intelligence (AI) machine learning models, and computer programs, all of which are executable by the processor 110. The various types of ROM and RAM may include any number, type and combination of computer readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, Blu-ray disk, a universal serial bus (USB) drive, a solid state drive (SSD), or any other form of storage medium known in the art. The memory 120 is a tangible storage medium for storing data and executable software instructions, and is non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The memory 120 may store software instructions and/or computer readable code that enable performance of various functions. The memory 120 may be secure and/or encrypted, or unsecure and/or unencrypted.

The processor 110 and the memory 120 may include or have access to an AI engine or module, which may be implemented as software that provides artificial intelligence and machine learning algorithms, such as neural network modeling, described herein. The AI engine may reside in any of various components in addition to or other than the processor 110, such as the memory 120, an external server, and/or the cloud, for example. When the AI engine is implemented in a cloud, such as at a data center, for example, the AI engine may be connected to the processor 110 via the internet using one or more wired and/or wireless connection(s).

The user interface 112 is configured to provide information and data output by the processor 110 and/or the memory 120 to the user and/or to provide information and data input by the user to the processor 110 and/or the memory 120. That is, the user interface 112 enables the user to enter data and to control or manipulate aspects of the processes described herein, and to control or manipulate aspects of the X-ray imaging. The user interface 112 also enables the processor 110 to indicate the effects of the user's control or manipulation to the user.

All or a portion of the user interface 112 may be implemented by a graphical user interface (GUI), such as GUI 118 on a touch screen 116 of the display 114, for example. The user interface 112 includes push buttons operable (pushed) by the user to initiate various commands for manipulating the displayed image, making measurements and calculations, and the like during an imaging session (e.g., cone beam computed tomography “CBCT” or other X-ray examination) or at any point during an interventional procedure performed under X-ray guidance. The push buttons may be displayed by the GUI 118 on the touch screen 116, or may be physical buttons, for example. The user interface 112 may further include any other compatible interface devices, such as a mouse, a keyboard, a trackball, a joystick, microphone, a video camera, a touchpad, or voice or gesture recognition captured by a microphone or video camera, for example.

The display 114 may be any compatible monitor for displaying X-ray images, radiation shield positions, and other information, such as a computer monitor, a liquid crystal display (LCD), an organic light emitting diode (OLED), a flat panel display, or a solid-state display, for example. The display 114 includes the touch screen 116 and the GUI 118 to enable the user to interact with the displayed images and features, as discussed above.

The X-ray imaging system 130 includes an X-ray source 131 and an X-ray detector 132 connected in a fixed relationship to one another on a C-arm 133. The X-ray source 131 emits ionizing radiation, according to settings, such as dosage, frame rate, exposure time, and beam collimation, for example, that travels through a portion of the patient anatomy. The X-ray detector 132 receives the X-ray radiation that has traveled through patient anatomy and acquires X-ray images in response that enable visualization of the internal anatomy of the patient 155 on an operating table 156 in images, such as X-ray images, fluoroscopy sequences, CBCT images, for example. When contrast is injected into the vasculature of the patient 155 during X-ray image acquisition, the visualization of the vascular anatomy of the patient 155 is enabled in images, such as Digital subtraction angiography (DSA) images and three-dimensional rotational angiography (3DRA) images, for example. The C-arm 133 is maneuverable for changing the location of the X-ray source 131 relative to the patient 155 to accommodate a variety of different viewing angles for the images. The X-ray imaging system 130 may be a fixed C-arm X-ray system mounted in the procedure room or a mobile C-arm X-ray system that is portable.

More particularly, the X-ray source 131 is positioned relative to a region of interest (ROI) 157 of the patient 155 to obtain images of the patient's anatomy, for example, during an interventional procedure being performed by the clinician 150. The ROI 157 may be an operation site or access site for the interventional procedure, for example. An X-ray imaging interface 135 interfaces the X-ray imaging system 130 with the control unit 105 to convert X-ray image data to a format compatible with the processor 110 and/or to communicate X-ray imaging system information (e.g., encoded C-arm position) to the control unit 105. The control unit 105 sends control signals to the C-arm 133 for controlling placement of the X-ray detector 132 relative to the patient 155, and for controlling image acquisition including timing, frame rate, power and other imaging parameters. The control unit 105 also receives and processes the X-ray image data from the X-ray detector 132 in response to operation of the X-ray source 131. The clinician 150 (e.g., interventionalist or radiology technician) may control operations of the X-ray imaging system 130 through the user interface 112, although it is understood that control of the X-ray imaging system 130 may be partially or entirely performed through a separate control unit, without departing from the scope of the present teachings.

The shield placement system 140 includes the radiation shield 142 and a control interface (IF) 144 configured to control movement of the radiation shield 142. The radiation shield 142 is formed of radiation shielding material(s), such as lead glass, for example. In an embodiment, the radiation shielding material is transparent so that placement of the radiation shield 142 for maximizing protection from radiation does not obstruct the visual line of sight of the clinician 150 to the ROI 157. Alternatively, the transparent radiation shielding material may include transparent polymeric sheets coated with radiation absorbing material. The transparent polymeric sheets allow flexibility of the shape (e.g., concave or convex) of the radiation shield 142 or portions of the radiation shield 142. The radiation absorbing material may include lead or non-toxic alternatives to lead, such as tungsten, bismuth or barium sulfate, for example. The transparent polymeric sheets additionally may be coated with a thin film of self-cleaning, self-sterilizing, antimicrobial polymers to help keep the environment sterile and to avoid manual cleaning requirements that may damage the radiation absorbing layers. In an embodiment, the radiation shield 142 may include multiple interconnected portions that can fold or slide over each other to allow for changeable coverage area of the radiation shield 142.

Optimizing protection of the clinician 150 from X-ray radiation includes protecting the clinician 150 from X-ray radiation that interacts with surfaces directly after being emitted from the X-ray source 131 and X-ray radiation reflected from surfaces of the entities present in the procedure room, such as the clinician 150, the patient 155, the operating table 156, and other people and objects. After being emitted from the X-ray source 131, X-ray radiation that interacts directly with surfaces may be referred to as “direct radiation,” and radiation reflected from surfaces that subsequently interacts indirectly with surfaces of entities present in the procedure room may be referred to as “scattered radiation.” The direct radiation and scattered radiation may be collectively referred to as “X-ray radiation,” and the patterns or levels of exposure to surfaces of entities in the procedure room from the combined effects of direct and scattered radiation may be referred to as “X-ray radiation patterns” or simply “radiation patterns”. The control IF 144 includes one or more motors (e.g., servo motors), actuators and/or other drive devices configured to move the radiation shield 142 and/or one or more portions of the radiation shield 142. In an embodiment, the radiation shield 142 may be encoded such that the exact position of the radiation shield 142 is available to the control unit 105. Alternatively, the radiation shield 142 may have external positioning devices attached, such as electromagnetic (EM) sensors or optical signal generators, for example, that are detectable by an external sensor to obtain position data, such as first sensor 145 discussed below. Or, the external sensor may be a camera configured to acquire images of the radiation shield 142 to obtain the position data, again as discussed below with regard to the first sensor 145.

The radiation shield 142 is adjustable to optimize protection of the clinician 150 (and other people in the procedure room), which includes reducing exposure of the clinician 150 to the X-ray radiation originating from the X-ray source 131 to the minimum extent possible without inhibiting access by the clinician 150 to the ROI 157 of the patient 155. That is, the position of the radiation shield 142 is adjustable to minimize the amount of radiation received by the clinician 150 as well as by the radiation-sensitive parts of the patient 155, without obscuring the clinician's view of the ROI 157 (e.g., intervention site) and/or without inhibiting the access to the ROI 157. The position the radiation shield 142 may also be adjusted according to the position the C-arm 133 in order to prevent the radiation shield 142 from obscuring the field of view of the X-ray imaging system 130.

As used herein, “position” refers to the location (e.g., Cartesian coordinates) and orientation (e.g., rotational coordinates) of the radiation shield 142. In various embodiments, the radiation shield 142 may be configurable (flexible), meaning that the coverage area (e.g., size and shape) of the radiation shield 142, in addition to the location and orientation, may be altered to further refine the shielding provided by the radiation shield 142. For example, the radiation shield 142 may include multiple interconnected sections and panels. At least one section of the interconnected sections may be configured to fold and unfold relative to another section in order to change the coverage area of the interconnected sections. Also, at least one section of the interconnected sections may include interconnected panels, where at least one panel of the multiple panels may be configured to slide relative to another panel in order to further change the coverage area of the interconnected sections. Accordingly, when the radiation shield 142 is configurable, “position” refers to the location, orientation and configuration of the radiation shield.

The radiation shield 142 is adjustable to accommodate changing variables within the procedure room during the interventional procedure, such as the location and orientation of the C-arm 133 which controls the location and orientation of the X-ray source 131, the size and position of the patient 155, the number and locations of the clinician 150 and other medical personnel relative to the X-ray source 131, for example. The shield placement system 140 is useful for any of various types of procedures, such as imaging procedures and any interventional endovascular and endobronchial procedures, for example.

The shield placement system 140 further includes a first sensor 145 configured to provide first position data indicating positions of the clinician 150, the patient 155 and objects in the procedure room (e.g., the radiation shield 142, the operating table 156). The first sensor 145 may be a camera, such as RGB or an RGB-D camera, for example, that provides image data and depth information regarding the objects within its field of view, including the clinician 150, the patient 155 and various objects, for example. In an alternative embodiment, the first sensor 145 may include one or more position tracking devices, such as an electromagnetic (EM) detector or an optical sensor, for example. In this case, corresponding positioning devices, such as EM sensors or optical signal generators, respectively, would be attached to the people and objects in the field of view of the first sensor 145 for which first position data is desired. For example, the clinician 150 may have EM sensors on a wearable badge, a wristband, garments, or the like.

The processor 110 receives the first position data from the first sensor 145 via a first sensor interface 146. When the first sensor 145 is a camera, for example, the first position data include image data. The first sensor interface 146 enables the first sensor 145 to send the first position data to the processor 110 and to receive control commands from the processor 110 (e.g., adjusting imaging parameters, triggering image acquisitions). The processor 110 determines positions of the people and objects in three-dimensions by applying any compatible position determination algorithm to the first position data, as would be apparent to one skilled in the art. For example, the processor 110 determines the positions of the clinician 150, the patient 155, and the operating table 156, as well as the radiation shield 142, using the first position data provided by the first sensor 145. The processor 110 may further determine the locations of the ROI 157 and any sensitive anatomical regions 158 of the patient 155 using the first position data. The sensitive anatomical regions 158 may include pelvic regions of reproductive age patients, for example.

The processor 110 may also receive second position data from the first sensor 145 indicating the position the X-ray source 131, in substantially the same manner discussed above. The processor 110 determines the second position of the X-ray source 131 in three-dimensions by applying any compatible position determination algorithm to the second position data, received from the first sensor 145. Alternatively, the shield placement system 140 further includes a second sensor 147 incorporated in the C-arm 133 of the X-ray imaging system 130. The second sensor 147 is configured to provide the second position data, indicating the position of the X-ray source 131, to the processor 110 via the X-ray imaging interface 135. In embodiments, the second sensor 147 includes an internal encoder configured to receive motion data indicating movement of the X-ray source 131 and/or the C-arm 133 during operation, and to translate the motion data to provide the second position data.

In the depicted embodiment, the memory 120 includes inter alia an X-ray radiation model module 121 for applying an X-ray radiation model and a shield positioning model module 122 for applying a shield positioning model. The X-ray radiation model may comprise a mathematical model (also known as physics based model) that calculates X-ray radiation patterns in the procedure room. The calculations may include the impact of direct radiation from the X-ray source and/or scattered radiation from the direct radiation reflected from entities in the procedure room for any given angle of the C-arm 133 and may include the position of the X-ray source and the entities in the procedure room. The X-ray radiation model may analytically calculate radiation patterns in the procedure room or predict radiation patterns in the procedure room using machine-learning (e.g., using a neural network). The X-ray radiation model receives as input the positions of the clinician 150, the patient 155, the operating table 156, and any other entities in the room that may reflect the X-ray radiation (e.g., walls, ceiling, tables), from the first position data provided by the first sensor 145, the position (pose information) of the X-ray source 131 from the second position data provided by the first sensor 145 or the second sensor 147, and the X-ray related settings of the X-ray imaging system 130. Such settings may include dosage, frame rate, exposure time, and collimation of the X-ray radiation emitted by the X-ray source 131, for example.

In embodiments using machine-learning, the radiation model (e.g., neural network) may be previously trained, using a processor before being applied for an actual procedure. The training may include receiving historic data (actual and/or simulated) including previous positions of entities (e.g., clinicians, patients, operating tables, and the like) based on previous first position data generated by a sensor during corresponding previous procedures, and including previous positions of the X-ray source 131 based on previous second position data generated by a sensor during the previous procedures. The training may further include receiving other historic data, such as previous X-ray system settings (e.g., dosage, frame rate, exposure time, and collimation of the X-ray radiation emitted by the X-ray source, etc.) during the previous procedures and/or previous measured radiation at locations within the procedure room during the previous procedures. The training may correlate relationships between the previous positions of the entities; the previous imaging source positions; the previous X-ray system settings, and/or the previous measured radiation at locations in the procedure room during the previous procedures to generate the trained radiation model. The trained radiation model is configured to predict and output estimated radiation patterns in a procedure room based on input of the current positions of one or more entities and the X-ray source in a procedure room and, optionally, the current X-ray system settings.

The X-ray radiation model outputs an estimated radiation pattern that includes the impact of direct radiation and scattered radiation and indicates how the radiation may spread when the X-ray source 131 is turned on at the particular settings. The direct radiation indicates radiation doses delivered to various entities in its path, including the clinician 150 and the patient 155, for example. The scattered radiation indicates how the X-ray beam interacts with (is reflected by) the various entities in its path, and allows computation of estimated radiation doses that may be delivered to entities outside the direct path of the X-ray beam emitted by the X-ray source 131. The radiation pattern may be visualized as scattered points (e.g., scatter plot or swarm plot), the densities of which correspond to the dosage levels of the scattered radiation, or as contour lines defining corresponding areas having the same dosage.

The control unit 105 is further configured to determine an optimal position of the radiation shield 142 during the procedure that minimizes exposure of the clinician 150 to the X-ray radiation originating from the X-ray source. In particular, the processor 110 applies the X-ray radiation model to the position of the clinician 150 indicated by the first position data, and the position of the X-ray source 131 indicated by the second position data, to determine the radiation pattern of the X-ray radiation emitted by the X-ray source 131 and reflected by entities in the procedure room. Such entities include but are not limited to the clinician 150, the patient 155, the radiation shield 142, and the operating table 156. The processor 110 also applies the shield positioning model to the position of the clinician 150 indicated by the first position data, position of the X-ray source 131 indicated by the second position data, and the determined radiation patterns to determine the optimal position of the radiation shield 142. As discussed above, the position of the radiation shield 142 refers to its location and orientation, as well as its configuration when applicable.

The processor 110 may visualize the optimal position of the radiation shield 142 on the display 114. This enables a user (e.g., the clinician 150) to manually move the radiation shield 142 to the optimal position by referencing the display 114. The user may manually move the radiation shield by grasping the radiation shield itself or by grasping handles or holds mounted on radiation shield 142 and physically maneuvering the radiation shield 142 to the optimal position. Alternatively, or in addition, the user may manually move the radiation shield 142 using controls at the user interface 112 and/or the GUI 118 on the touch screen 116, to send commands to electronically control the movement of the radiation shield 142 via the control IF 144. In this case, the commands entered by the user control operations of the motors, actuators and/or other drive devices in the control IF 144 configured to move the radiation shield 142 and/or portion(s) of the radiation shield 142. In an embodiment, the processor 110 visualizes the optimal position of the radiation shield 142 together with a current position of the radiation shield 142 on the display 114. This gives the user visual perspective on how to maneuver the radiation shield 142 from its current position to the optimal position.

In an embodiment, the control unit 105 further includes a projector configured to project directions in which to move the radiation shield 142 to its optimal position onto a surface under control of the processor 110. Projecting the directions allows the user to easily observe and follow the directions to place the radiation shield 142 in the optimal position. For example, the directions may be projected onto the radiation shield 142 itself, and may include straight arrows indicating directions in which the radiation shield 142 should be translated and curved arrows near the edges of the radiation shield 142 indicating which edges should be rotated and in which directions in order to position the radiation shield 142 optimally. The control unit 105 may likewise include an augmented reality (AR) display, e.g., included in AR glasses worn by the user, which may similarly display arrows indicating directions in which to translate and/or rotate the radiation shield 142 to position it in the optimal position.

In an embodiment, the processor 110 automatically maneuvers the radiation shield 142 to the optimal position by controlling the operations of the motors, actuators and/or other drive devices in the control IF 144 in order to drive the radiation shield 142 into the optimal position. In this case, the memory 120 includes a shield driver module 123, which receives current position data regarding the current position of the radiation shield 142, e.g., from encoding of the radiation shield 142 or from the first sensor 145, for example, and optimal position data regarding the optimal position of the radiation shield 142 from the shield positioning model module 122. The shield driver module 123 then calculates movement data, including vectors and angles of rotation, for example, indicating the desired movement of the radiation shield 142 from the current position to the optimal position, and provides the movement data to the control IF 144 to be implemented. When the radiation shield 142 is configurable, the movement data may further include differences between the size and shape of the radiation shield 142 in its current configuration and the size and shape of the radiation shield 142 in its optimal configuration in the optimal position. In this case, the movement data indicates the arrangements of the sections and panels in the current configuration and the optimal configuration, respectively, so that any differences can be identified and implemented to drive the radiation shield into the optimal position. The shield driver module 123 may receive feedback regarding intermediate positions of the radiation shield 142 during the moving process for updating the movement data, if necessary.

As discussed above, the control IF 144 may include motors, actuators and/or other driving devices that operate in response to the commands from the control unit 105. The commands from the control unit 105 to the control IF 144 may be provided through electrical wiring or using a wireless system, such as Bluetooth or Wi-Fi, for example. In an embodiment, user commands may be transferred to the control unit 105 via the user interface 112 through hand gestures or voice recognition protocols, and/or through wired or wireless remote control, for example. Also, the control unit 105 may include a separate robot controller for controlling operation of the control IF 144, as would be apparent to one skilled in the art. In an embodiment, movement of the radiation shield 142 may be a combination of automatic and manual controls, without departing from the scope of the present teachings.

In an embodiment, the user is provided the opportunity via the user interface 112 and/or the GUI 118 to accept or reject the optimal position of the radiation shield 142. When the user rejects the optimal position, the processor 110 may again apply the shield positioning model to the first and second positions and the determined radiation patterns to determine another optimal position of the radiation shield 142, after changing at least one parameter of the first and second positions and/or of the shield positioning model itself. The parameter(s) may be changed by the user or automatically by the processor 110. Manual changes in parameters may include user indicating via a touch screen interface areas to avoid (e.g., areas where the radiation shield 142 may inhibit the clinician's access to the ROI 157) or areas to further protect (e.g., radiation-sensitive parts of the patient 155). Automatic changes in parameters may include, for example, optimizing radiation shield position for the clinician that is second closest to the X-ray source instead of the clinician closest to the X-ray source.

The shield positioning model is configured to estimate exposure of the clinician 150 to X-ray radiation from the X-ray source 131 in the position indicated by the second position data based on the estimated radiation patterns output by the X-ray radiation model, and to estimate the optimal position of the radiation shield 142 that minimizes the exposure of the clinician 150 to the X-ray radiation from the X-ray source 131. The shield positioning model may receive as input the position of the clinician 150 determined from the first position data, the position of the X-ray source 131 determined from the second position data, and the radiation patterns output by the X-ray radiation model. Based on this input, the shield positioning model compute and outputs the optimal position of the radiation shield 142 that minimizes radiation exposure to the clinician 150 by, for example, minimizing overlap between a position of the clinician and a given level of radiation exposure in the radiation patterns. In some embodiments, the radiation shield includes moveable interconnected sections and panels and the shield positioning model computes the positions for these interconnected sections and panels to provide the optimal position of the radiation shield. In some embodiments, the shield positioning model may comprise a neural network algorithm, such as an artificial neural network (ANN) algorithm, a convolutional neural network (CNN) algorithm, or a recurrent neural network (RNN) algorithm. In other embodiments, the shield positioning model may use a lookup table, such as a relational database, that maps the model input, including locations of the X-ray source, clinicians, patients, tables, etc. in the procedure room and corresponding radiation patterns, to a predetermined optimal position for the radiation shield. In yet other embodiments, the model may analytically compute the optimal position of the radiation shield from the model input based on known and/or simulated relationships between locations of the X-ray source, clinicians, patients, tables, etc. in the procedure room and corresponding radiation patterns.

In an embodiment, the shield positioning model computes the optimal position of the radiation shield 142 to take into consideration the location of the ROI 157 so that the radiation shield 142 does not block visual and/or physical access to the ROI 157 by the clinician 150. In another embodiment, the shield positioning model computes the optimal position of the radiation shield 142 to instead or also takes into consideration the locations of sensitive anatomical regions 158 so that the radiation shield 142 prioritizes protection at this location, in addition to protecting the clinician 150.

In some embodiments using supervised learning, any sensitive areas can be identified during training using bounding boxes or other region of interest indicators. For instance, areas to avoid or prioritize in computing the optimal position of the radiation shield 142 may be indicated by the clinician 150 or other user (e.g., by outlining areas to avoid in red, and outlining areas to prioritize in green on user interface 112) in training data, allowing the shield positioning model to optimize its weights during supervised training using loss functions that assign higher errors to shield positions overlapping with areas to avoid and lower errors to shield positions overlapping with areas to prioritize. This allows the shield positioning model, for instance, to prioritize areas indicated by the user (e.g., in green) during inference or application of the shield positioning model. For example, the user interactively indicate on the touchscreen 116 an area to prioritize, allowing the shield positioning model's output of the optimal shield position to be updated. Alternatively, the system may automatically infer which patients require additional anatomical regions to be protected. For example, in an embodiment, the electronic health record (EHR) data of the patient may additionally be input into the shield positioning model, allowing the shield positioning model to access information, including patient age and sex, and to optimize its weights during supervised training such that shield positions reducing radiation to the identified pelvic areas of women of child-bearing age are prioritized, for example. By changing the weights of the shield positioning model additionally based on the patient EHR data (including age, sex, and pregnancy status) during the training, the shield positioning model learns from its parameters that features associated with identified regions of the patient anatomy must be protected. Therefore, the shield positioning model will learn that for a pregnant woman or woman of child-bearing age, for example, the area around the abdomen and pelvic region must be protected, and will predict radiation shield positions that prioritize the protection of these regions for the relevant patients during inference.

The memory 120 may also store information about the procedure, such as “target procedure” identifying the type of procedure, “target anatomy” identifying the part of the patient's body that is the subject of the procedure, “procedure phase” identifying the part of a multifaceted procedure being performed, and/or “access site” identifying a location on the patient's body at which devices are inserted into the patient body. Using this information, the processor 110 may automatically identify regions around the patient 155 that should be avoided or excluded from optimization of the position of the radiation shield 142 in substantially the same manner the location of the ROI 157 is avoided. For example, when the clinician 150 is gaining access at a femoral site of the patient 155, the processor 110 may exclude regions around the patient's leg from optimization so as to avoid obscuring the proper access of the clinicians' hands to the access site.

In addition, the clinician 150 may enter via the user interface 112 and/or the GUI 118 ranges in the procedure room to exclude from optimization based on his or her personal comfort level, so that the optimal position of the radiation shield 142 does not end up within these ranges. The control unit 105 may learn user preferences of the clinician 150 by keeping track of excluded regions and optimal positions of the radiation shield 142 over time and/or after multiple procedures during which suggested optimal positions of the radiation shield 142 have been accepted or rejected by the clinician. The user preferences may be input to the shield positioning model to personalize the determination of the optimal position of the radiation shield 142 for a given user. For example, the user preferences may be implemented by weighting the loss function such that positions of the radiation shield 142 similar to those previously accepted by the clinician 150 generate lower errors than those not previously accepted or otherwise expressly rejected by the clinician 150, so that preferred shield positions contribute less to the accumulated error while rejected shield positions contribute more to the error, prompting the shield positioning model to prefer shield positions previously accepted by the clinician 150.

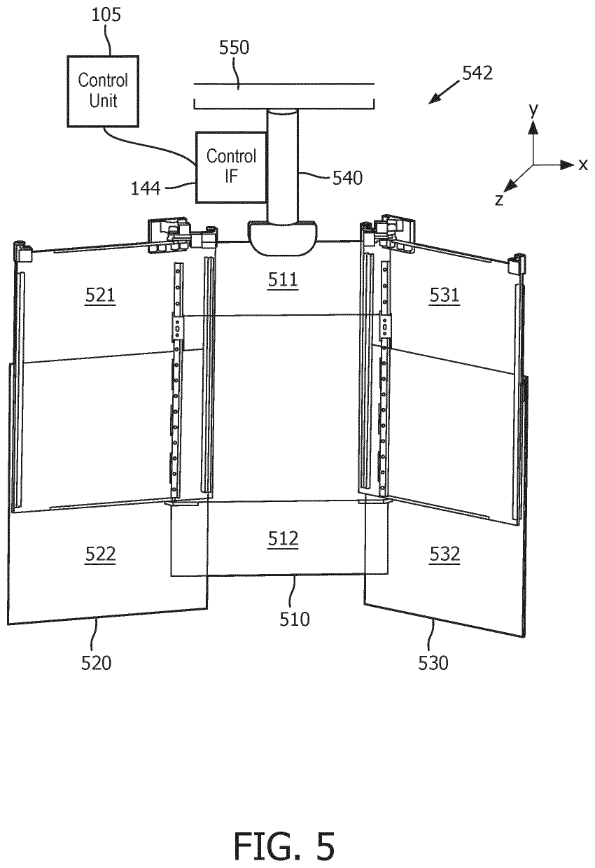

The shield positioning model (e.g., neural network) may be previously trained by a processor before being implemented for an actual procedure. The training may include receiving historic data (actual and/or simulated) including previous positions of entities (e.g., clinicians, patients, operating tables, and the like) based on previous first position data generated by a sensor during corresponding previous procedures, and including previous positions of the X-ray source 131 based on previous second position data generated by a sensor during the previous procedures. The training may further include receiving other historic data, including previous estimated radiation patterns in the procedure room during the previous procedures. For example, the training may include receiving previous estimated radiation patterns analytically calculated or predicted by the X-ray radiation model from the previous first and second position data. The training may also include receiving further historic data, including previous positions of a radiation shield 142 in the procedure room during the previous procedures and may include the previous measured radiation to the entities at the previous positions of the radiation shield 142. In some embodiments, the radiation shield has moveable interconnected sections and panels, such as radiation shield 542 described with respect to FIG. 5, and the previous positions of the radiation shield includes (i) position of at least one section of the interconnected sections of the radiation shield relative to the other sections of the interconnected sections of the radiation shield and/or (ii) position of at least one panel of the panels of the radiation shield relative to other panels of the radiation shield. The previous data may be retrieved from a database or other memory (e.g., memory 120), for example, accessible by the processor 110.

The training may correlate relationships between the previous positions of the entities (clinicians, patients, operating tables, etc.); the previous imaging source positions; the previous estimated radiation patterns; the previous radiation shield positions (e.g., in some embodiments, including position of sections and/or panels thereof); and/or the previous measured radiation to entities at the previous radiation shield positions during the previous procedures to generate a trained shield positioning model. The trained shield positioning model is configured to predict and output optimal positioning of the radiation shield (e.g., in some embodiments, including optimal position of sections and/or panels thereof) that minimizes radiation exposure to a clinician based on input of the current positions of the entities and the imaging source in the procedure room and the current estimated radiation patterns in the procedure room.

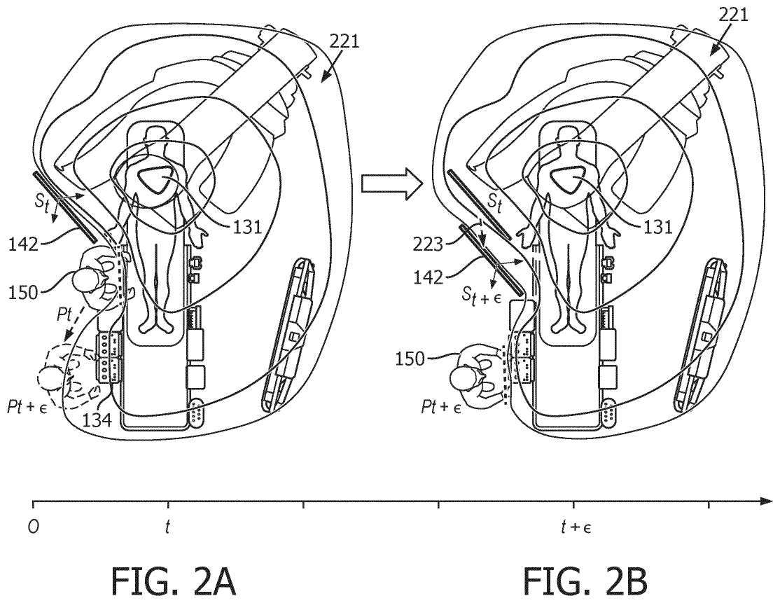

The training may include one or more loss functions implemented to predict the optimal position of the radiation shield according to predetermined optimization criteria. For example, the loss functions may be implemented to weigh or balance application of the previous data, such as the previous positions of clinicians, patients, operating tables, etc.; the previous imaging source positions; the previous estimated radiation patterns; the previous radiation shield positions; and/or the previous measured radiation to entities at the previous radiation shield positions during the previous procedures, according to predetermined optimization criteria. For example, one optimization criterion may be to minimize radiation exposure to the clinician 150 and/or other personnel standing closest to the X-ray source 131 using the radiation shield 142. In this embodiments, the loss function may be implemented to weight the previous data to predict the optimal radiation shield position to minimize the difference in pose between an estimated shield pose(s) and the principal plane of intersection (i) between a given level of radiation exposure (indicated by certain shadings on the heatmaps in FIGS. 2A and 2B or by contour lines indicating levels of radiation) and the clinician. The loss may be a difference between the three degrees of freedom (DoF) shield pose, estimated by the shield positioning model (two DoF in-plane translation, one DoF in-plane rotation) and the principal plane of intersection. For example, the loss function may apply the Euclidean distance between the two DoF translation components of the shield pose and the plane of intersection, and the angle between the one DoF in-plane rotation components of the shield pose and plane of intersection. Similar loss functions can be implemented for the radiation shield with greater than three DoF. When the radiation shield 142 is configurable, and thus able to slide along its height to change its vertical configuration for example, the translation components may include all three DoFs. The Euclidean distance between the translation components of shield poses may also be substituted by other distance measures, such as Riemannian distance, Geodesic distance, for example.

FIGS. 2A and 2B are schematic views of radiation shield placement minimizing radiation exposure to the clinician, according to a representative embodiment. Referring to FIG. 2A, the shield positioning model inputs first position information with regard to the position of the clinician 150 and the initial position of the radiation shield 142, second position information with regard to the position of the X-ray source 131 and/or the C-arm 133. The shield positioning model further inputs the estimated radiation patterns 221 output by the X-ray radiation model. Referring to FIG. 2B, as the clinician 150 moves into a higher radiation area, the shield positioning model outputs an optimal pose of the radiation shield 142 (indicated by the arrow 223) to minimize radiation exposure to the clinician 150. FIG. 2B also shows the shield pose(s) and the principal plane of intersection (i) between a given level of radiation exposure (indicated by certain shadings on the heatmaps representing the radiation pattern 221 in FIGS. 2A and 2B or by contour lines indicating levels of radiation), as determined by the X-ray radiation model, and the clinician 150.

More particularly, FIG. 2A shows that at time t, the clinician 150 is at position pt and the shield 142 is positioned at the optimal position st so that radiation to clinician 150 is minimized. However, radiation near the controls 134 is high, as indicated by the radiation pattern 221. Therefore, if the clinician 150 moves there at future time t+ϵ (as indicated by the transparent figure in FIG. 2A), then the radiation exposure to the clinician 150 would be high. In FIG. 2B, when the clinician 150 moves to the controls 134 at time t+ϵ, the optimal position of the radiation shield 142 is updated from sₜ to s_{t+ϵ} so that, again, radiation to clinician 150 is minimized.

As mentioned above, another optimization criterion may be to avoid either obscuring or inhibiting access to the ROI 157 on the patients in the previous procedures. This may be achieved by annotating regions to avoid in the training data relative to the ROI 157, and penalizing closeness of the estimated shield pose(s) to the annotated regions (a_{avoid}). In this case, the Euclidean distance between the annotated region (a_{avoid}) and the shield pose(s) may be maximized at the same time that distance between the shield pose(s) and the principle plane of intersection (i) is minimized, discussed above.

Another optimization criterion may be to protect the sensitive anatomical regions 158 identified on the patients in the previous procedures from direct radiation and/or scatter radiation without obscuring the ROI 157 or other critical regions. Similar to the discussion above, the sensitive anatomical regions 158 to avoid may be annotated in the training data. In this case, the Euclidean distance between the annotated regions (a_{include}) and the shield pose(s) may be additionally minimized during optimization while penalizing shield poses that may fall within the field of view to be imaged. This may be done by modeling the X-ray beam generated from the X-ray source 131 given current X-ray settings (e.g., dosage, collimation) using the X-ray radiation model, and evaluating whether the radiation shield 142 intersects the X-ray beam in various positions. When the radiation shield 142 intersects the X-ray beam traveling toward the field of view to be imaged, the corresponding positions are rejected since they fall within the field of view. On the other hand, when the radiation shield 142 intersects the X-ray beam traveling toward a_{include}, the corresponding positions are prioritized.

Notably, the training of the shield positioning model is described above with reference to the system 100, which is the same system used for subsequently performing the procedure for which the X-ray radiation model and the trained shield positioning model are used. It is understood, however, that training data for training the shield positioning model may be obtained from other systems, similarly configured to the system 100, without departing from the scope of the present teachings.

Also, in an embodiment, the training of the shield positioning model may take place in a simulation environment, e.g., executable by the processor 110. The simulation environment generates data in the form of rendered scenes of the procedural environment where various parameters can be varied, such as X-ray image settings and geometry, and positions of clinicians and other entities, for example, in order to generate large amounts of simulated data. The optimal pose of the radiation shield 142 may be modeled in the simulation based on radiation propagation from the X-ray radiation model and other physical characteristics. The radiation propagation (or mapping) may be computed using physics-based simulation, such as a Monte Carlo simulation, for example, or data driven solutions to estimate volumetric heatmaps around the ROI 157 (e.g., the interventional access site). In this case, the loss function may include the distance between the estimated pose of the radiation shield 142 and the ground-truth pose. As described above, depending on the design of the radiation shield 142, one to six or more DoF are considered for the radiation shield 142. The simulated data may also be used in combination with real data, and may be used to evaluate the shield placement system 140 and to quantify how much reduction in radiation exposure is achieved.

In an embodiment, the system 100 may further provide collision prevention using the first and second position data, and/or one or more distance measurement sensors, such as optical, acoustic, capacitive, inductive and/or photoelectric sensors, for example, arranged in the procedure room and/or on the radiation shield 142, and configured to measure distances between the radiation shield 142 and the clinician 150, patient 155, the operating table 156 and other equipment in the procedure room when the radiation shield 142 is being maneuvered. The measurement information may be provided to the control unit 105, which initiates an alarm whenever one of the measured distances becomes less than a predetermined safety threshold distance. The control unit 105 may also block further motion and/or configuration of the radiation shield 142 until the measured distance is again outside the predetermined safety threshold distance. This assures the safety of the clinician 150 and the patient 155, as well as the security of the radiation shield 142 during operation.

In another embodiment, the system 100 may include one or more radiation shields in addition to the radiation shield 142. In this case, the optimal positions of each of the radiation shields (including the radiation shield 142) relative to the clinician 150 are determined using the X-ray radiation model and a shield positioning model, as discussed above. The first position data acquired by the first sensor 145 would further include data regarding the respective positions of the radiation shields. These data are input to the X-ray radiation model (e.g., provided by shield positioning model module 122), which factors the positions into estimating the radiation pattern of the X-ray radiation originating from the X-ray source 131, since each radiation shield is an object that reflects the X-ray radiation to be considered in determining the optimal position of each of the other radiation shield(s). The optimal position of each of the radiation shields is then determined by applying the shield positioning model (e.g., provided by shield positioning model module 122) to the position of the at least one clinician, the position of the X-ray source, and the estimated radiation patterns, as discussed above with regard to the radiation shield 142. Also, the collision prevention discussed above may be incorporated into the system having multiple radiation shields in order to prevent collisions and/or overlapping optimal positions between radiation shields.

In an embodiment involving multiple radiation shields, loss between each radiation shield and personnel in the procedure room, including the clinician 150, may be computed in such a way that the position of each radiation shield is compared only with the positions of the personnel closest to it. For example, the personnel present in the procedure room may be clustered into n clusters respectively corresponding to the radiation shields, where n is the number of radiation shields (including the radiation shield 142). The processor 110 then computes the loss between the position of each radiation shield and the positions of the personnel in the corresponding cluster to determine the optimal position when applying the shield positioning model. As the personnel move about, the make-up of the n clusters may change, in which case the optimal position for each of the radiation shields may be updated. Alternatively, the position of each radiation shield may be compared with the n-th closest clinician to a level of radiation exposure in the radiation pattern. That is, the position of the first shield is compared to the person closest to a level of exposure, the second shield is compared to the person second closest to a level of exposure, and so on.

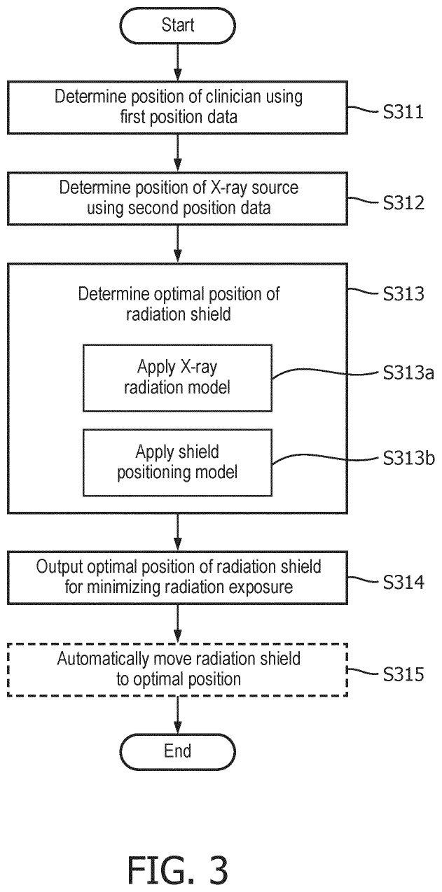

FIG. 3 is a flow diagram of a method for reducing exposure of at least one clinician to X-ray radiation from an X-ray source in a procedure room using a movable radiation shield, according to a representative embodiment. The method depicted in FIG. 3 may be implemented by the processor 110 of the control unit 105, executing instructions stored in the memory 120, for example.

Referring to FIG. 3, the method includes determining a position of the at least one clinician in the procedure room using first position data received from a first sensor (e.g., first sensor 145) in block S311, and determining a position of the X-ray source using second position data received from the first sensor or a second sensor (e.g., second sensor 147) in block S312. The X-ray source is configured to emit X-ray radiation (e.g., an X-ray beam) toward a patient (e.g., patient 155) in the procedure room in accordance with X-ray settings, such as dosage, frame rate, exposure time, and collimation of the X-ray radiation.

In block S313, an optimal position of a radiation shield (e.g., radiation shield 142) is determined, where the optimal position minimizes exposure of the at least one clinician to radiation from the X-ray source. Determining the optimal position of the radiation shield may include applying an X-ray radiation model (e.g., provided by X-ray radiation model module 121) that takes as input the position of the at least one clinician and the position of the X-ray source to estimate an X-ray radiation pattern in block S313a, including impact of direct radiation from the X-ray source and scattered radiation reflected from entities within the procedure room. Determining the optimal position may further include applying a shield positioning model (e.g., provided by shield positioning model module 122) that takes as input the position of the at least one clinician, the position of the X-ray source, and the estimated radiation pattern to determine the optimal position of the radiation shield in block S313b. The optimal position provides protection of the at least one clinician from the X-ray radiation. The optimal position may provide configurations for moveable interconnected sections and panels of the radiation shield to optimally position the radiation shield to provide protection of the at least one clinician from the X-ray radiation. In an embodiment, the X-ray radiation model may be further receive as input the X-ray settings of the X-ray source used to emit the X-ray radiation.

In block S314, the optimal position of the radiation shield is output to enable the radiation shield to be arranged in the optimal position. In an embodiment, the output of the optimal position of the radiation shield may include visualizing the optimal position of the radiation shield on a display (e.g., display 114), enabling the user (e.g., clinician 150) to manually adjust the radiation shield to the optimal position accordingly. In another embodiment, the optimal position of the radiation shield is visualized and displayed together with a current position of the radiation shield. This gives the user visual context when maneuvering the radiation shield from the current position to the optimal position. The user may manually move the radiation shield by physically touching and directing the radiation shield to the optimal position. Alternatively, or in addition, the user may manually move the radiation shield using controls at a user interface (e.g., user IF 112), including buttons provided by a GUI (e.g., GUI 118) on a touch screen (e.g., touch screen 116), to send commands controlling electronic movement of the radiation shield via a control interface (e.g., control IF 144). The GUI may be configured to provide feedback to the user while maneuvering the shield.

In block S315, the radiation shield is optionally moved automatically from the current position to the optimal position output in block S314. The radiation shield may be moved automatically by the control unit, which determines differences between the current and optimal positions of the radiation shield, and issues commands to the control interface to automatically drive the radiation shield from the current position to the optimal position based on these differences. As discussed above, the control interface includes motors, actuators and/or other driving devices that operate in response to the commands from the control unit. In this case, the control unit may include a robot controller, the operation of which would be apparent to one skilled in the art. In an embodiment, movement of the radiation shield may be a combination of automatic and manual controls, without departing from the scope of the present teachings.

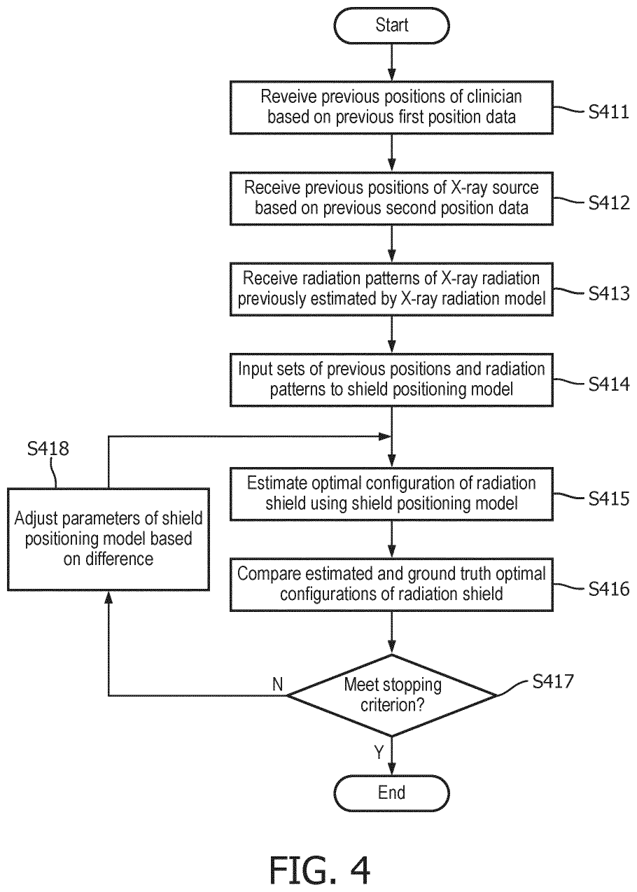

As discussed above, in embodiments in which the shield positioning models comprises a machine-learning model, the shield positioning model applied on block S313b is initially trained. FIG. 4 is a flow diagram of a method for training the shield positioning model for reducing exposure of the at least one clinician to X-ray radiation from the X-ray source, according to a representative embodiment. The method depicted in FIG. 4 may be implemented by the processor 110 of the control unit 105, executing instructions stored in the memory 120, for example, or another processor not part of the control unit. The training may be based on historic data that includes previous positions of clinicians, patients, and entities, previous positions of X-ray sources, and previously determined optimal positions of the radiation shields, respectively. The historic data may be data from actual procedures previously performed using the same system, including the same control unit, X-ray imaging system and shield positioning system, or using different but similar systems. Alternatively, all or part of the historic data may be simulated provided in a simulation environment.

Referring to FIG. 4, the method includes receiving previous positions of the at least one clinician based on previous first position data generated by a first sensor (e.g., first sensor 145) during previous procedures on respective patients in block S411, and receiving previous positions of the X-ray source based on previous second position data generated by the first sensor or a second sensor (e.g., second sensor 147) during the previous procedures in block S412.

In block S413, previous radiation patterns of the X-ray radiation emitted by the X-ray source estimated by the X-ray radiation model are received. The radiation patterns are based on the previous first position data and the previous second position data, respectively, thereby providing corresponding sets of the previous first position data, the previous second position data, and the estimated radiation patterns. The previous radiation patterns may include the impact of direct radiation and scattered radiation estimated by an X-ray radiation model (e.g., X-ray radiation model module 121), where the direct radiation indicates X-ray radiation emitted from the X-ray source and scattered radiation indicates X-radiation reflected from entities within the procedure room.

In block S414, sets of the previous first position data, the previous second position data, and the estimated radiation patterns are input to a shield positioning model, which is the shield positioning model being trained.

For each set of the previous first position data, the previous second position data, and the estimated radiation patterns, an optimal configuration of the radiation shield that minimizes exposure of the at least one clinician to the X-ray radiation from the X-ray source is predicted using the shield positioning model in block S415, and a difference between the predicted or estimated optimal configuration and a ground truth optimal configuration of the radiation shield in block S416. In some embodiments, the radiation shield includes interconnected moveable sections and panels and the optimal configuration predicted using the shield positioning model includes an optimal configuration of these sections and panels. In an embodiment, estimating the optimal configuration of the radiation shield in block S415 may include applying loss functions, which include one or more of minimizing radiation exposure to the clinician who is standing closest to the X-ray source (when there are multiple clinicians), avoiding obscuring or inhibiting access to a region of interest on the patients, and protecting an anatomical region of interest of the patients from X-ray radiation.

In block S417, it is determined whether a stopping criterion is met. For example, the stopping criterion may be when the difference between the estimated shield location and the ground truth optimal shield location is less than a predetermined threshold. When the stopping criterion has not been met (block S417: No), the training of the shield positioning model continues by adjusting the parameters of the shield positioning model based on the difference between the estimated optimal configuration and a ground truth optimal configuration of the radiation shield in block S418, and repeating the estimating the optimal configuration of the radiation shield using the shield positioning model with the updated parameters in block S415, and comparing the estimated optimal configuration and the ground truth optimal configuration of the radiation shield in block S416. When the stopping criterion has been met (block S417: Yes), the training process ends, resulting in a trained shield positioning model.

As mentioned above, the radiation shield 142 may be configurable, which means that the coverage area (e.g., size and shape) of the radiation shield 142, in addition to the location and orientation of the radiation shield 142, is alterable to further adjust the area of shielding provided by the radiation shield 142. In some embodiment, the radiation shield may include moveable interconnected sections and panels, which may be adjusted to adjust the area of shielding provided by the radiation shield 142.

FIG. 5 is a diagram of a system (e.g., system 100) including a perspective view of a configurable radiation shield, according to a representative embodiment. Generally, the radiation shield is configurable to accommodate changing variables within the procedure room to maximize protection from radiation during a procedure. As mentioned above, such variables include the size and shape of the procedure room, the location of the radiation source (e.g., X-ray source 131 and C-arm 133), the size and position of the patient, the number and locations of the medical personnel relative to the radiation source and/or the radiation source, for example.

Referring to FIG. 5, the system includes radiation shield 542, as well as the control unit 105 and the control IF 144, discussed above. The radiation shield 542 is placed within a procedure room for performing medical imaging and/or interventional procedures that require use of an imaging device that emits ionizing radiation, such as the X-ray source 131. In the depicted embodiment, the radiation shield 542 includes three interconnected sections, each of which includes a set of two vertically stacked panels (upper and lower), at least one of which is moveable relative to the other panel in a vertical direction (indicated by the y-axis).

More particularly, first (center) section 510 includes upper panel 511 and lower panel 512, second (left) section 520 includes upper panel 521 and lower panel 522, and third (right) section 530 includes upper panel 531 and lower panel 533. The first, second and third sections 510, 520 and 530 are formed of one or more radiation shielding material(s) in that each of the upper and lower panels 511, 512, 521, 522, 531 and 533 are formed of radiation shielding material(s), discussed above. In alternative embodiments, one or more of the first, second and third sections 510, 520 and 530 may include only a single panel of radiation shielding material.

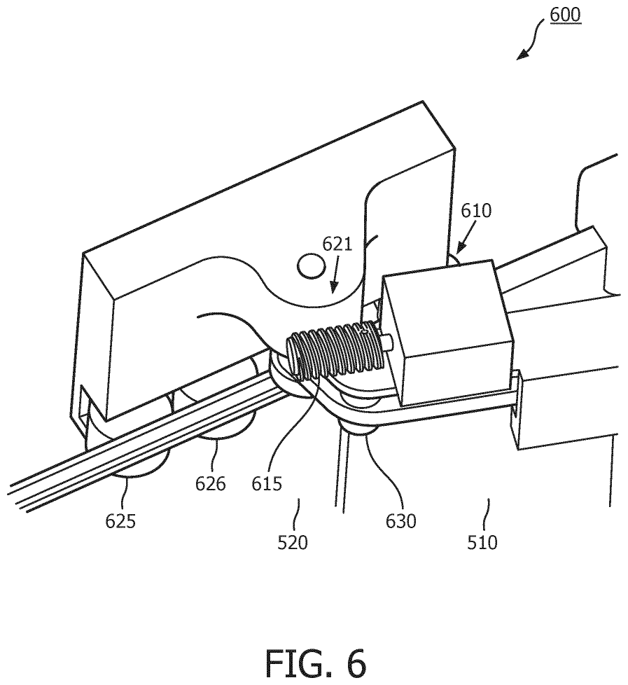

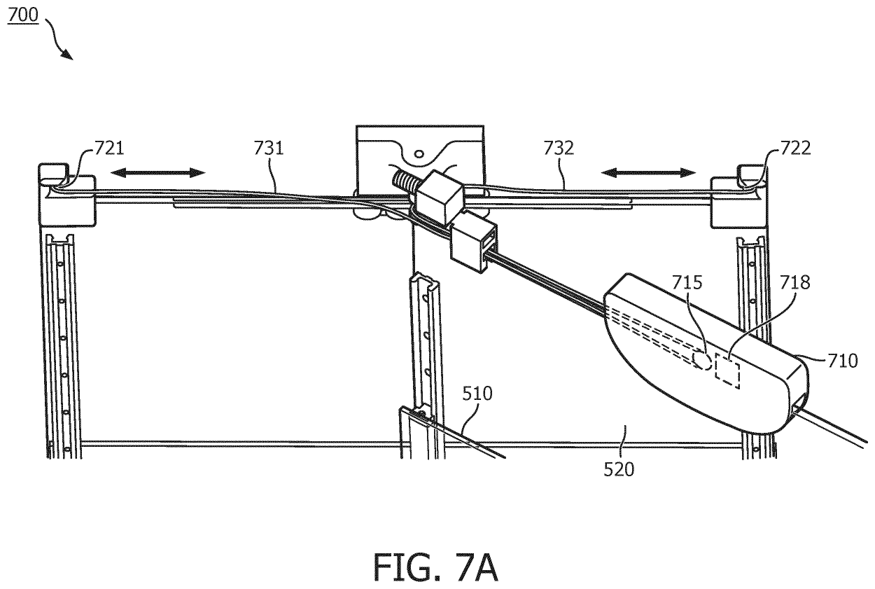

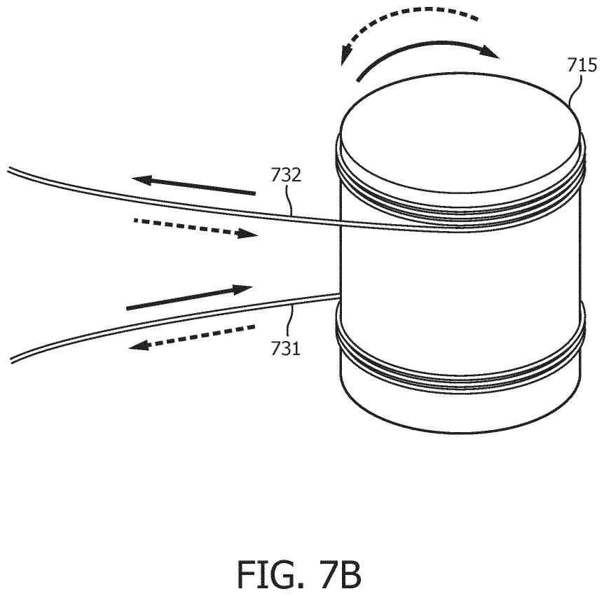

Each of the second and third sections 520 and 530 is configured to fold and unfold relative to the first section 510, for changing the width of the radiation shield 542 in a horizontal direction (indicated by the x-axis), making it narrower or wider. The folding and unfolding involves pivoting movements around the vertical axis (indicated by the y-axis). Examples of connections that enable the folding and unfolding movements of the second and third sections 520 and 530 are discussed below in reference to FIG. 6. Each of the second and third sections 520 and 530 may also be configured to move horizontally (translate) relative to the first section 510, for changing the width of the radiation shield 542 in the horizontal direction, making it narrower or wider. Examples of connections that enable the translation of the second and third sections 520 and 530 are discussed below in reference to FIGS. 7A and 7B.

In the first section 510, the upper and lower panels 511 and 512 are configured to move (e.g., slide or roll) vertically relative to one another for changing the length of the coverage area of the radiation shield 542 in the vertical direction. Likewise, the upper and lower panels 521 and 522 in the second section 520 and the upper and lower panels 531 and 532 in the third section 530 are each configured to move relative to one another in the vertical direction.

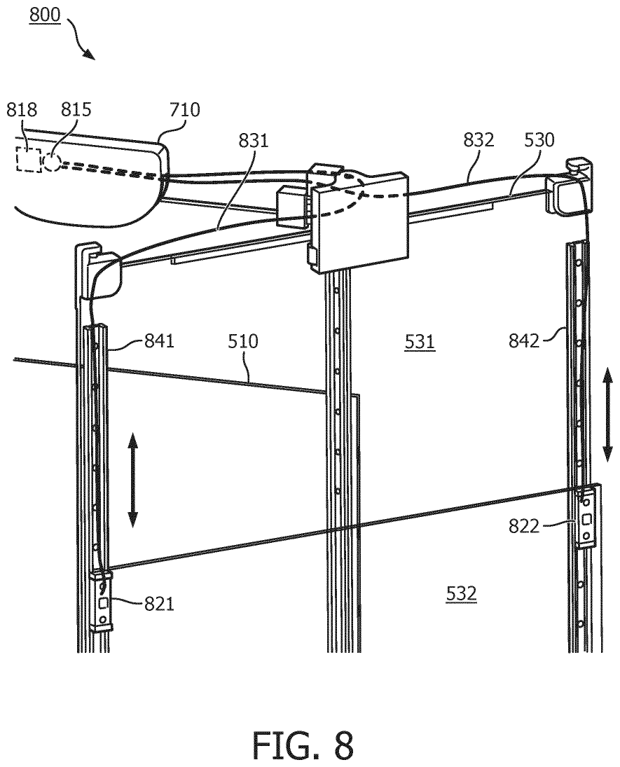

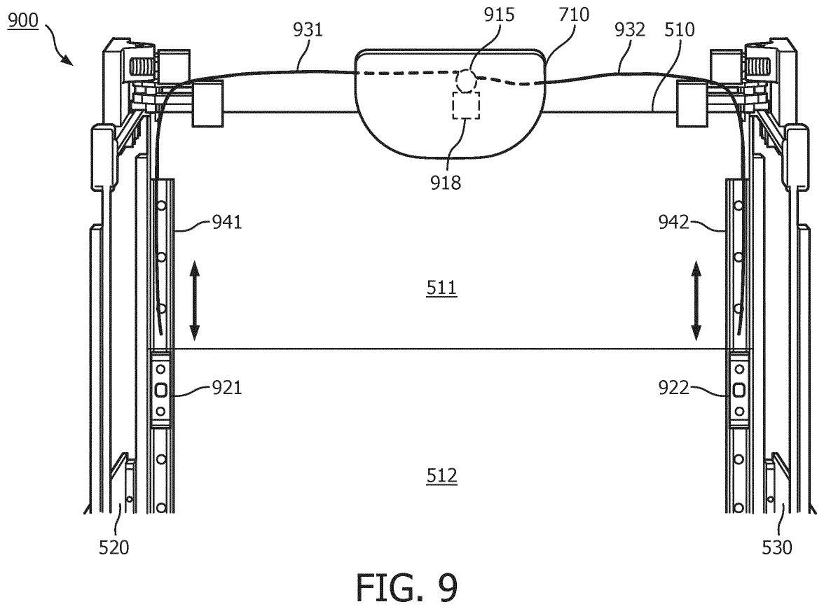

Examples of connections that enable the vertical movements of the panels are discussed below in reference to FIGS. 8 and 9. When the desired configuration of the radiation shield 542 is obtained, the first, second and third sections 510, 520 and 530 and/or the upper and lower panels 511, 512, 521, 522, 531 and 532 may be locked in place to maintain the overall shape of the radiation shield. For example, tension in cables used to adjust the upper and lower panels 511, 512, 521, 522, 531 and 532, discussed below, may hold them in place using stepper motors with sufficient holding torque. Alternatively, for example, an electric solenoid may be used in combination with mating holes to drive a pin in and out, to lock the upper and lower panels 511, 512, 521, 522, 531 and 532 in place. The solenoid pin may be used to lock movement in the vertical direction (indicated by the y-axis) as this will bear the weight of the panels.

Notably, although the depicted number of sections of the radiation shield is three, and the depicted number of radiation resistant panels per section is two, it is understood that more or fewer sections and/or more or fewer panels per section may be incorporated without departing from the scope of the present teachings, depending on factors such as room size and layout, type of procedure for which the procedure room is designed, the number and locations of medical personnel expected in the procedure room during the procedure, for example.

The radiation shield 542 further includes a mount 540 configured to moveably connect the three interconnected sections to a structure 550, such as the ceiling (as shown in the example of FIG. 5) and/or one or more walls of the procedure room. Alternatively, the structure 550 may be a free standing base to which the mount 540 is attached, where the free standing base itself may be repositioned around the procedure room using wheels or skids, for example. In the depicted embodiment, the mount 540 connects the first section 510, referred to as the primary panel, to the structure 550 from below. This leaves the second and third sections 520 and 530, referred to as the secondary sections, free to fold and unfold relative to the first section 510.

The mount 540 is attached to the first section 510 and/or the structure 550 in a manner that enables rotation of the first section 510 (and thus the second and third sections 520 and 530 connected to the first section 510) relative to the structure 550 around at least one of the y-axis, the x-axis or the z-axis, indicated in FIG. 5, where the y-axis is the vertical axis and the x-axis and the z-axis are horizontal axes perpendicular to one another. The first section 510 can spin around the y-axis, tilt left and right around the z-axis, and tilt front and back around the x-axis in any combinations of movements. The mount 540 may include any compatible mounting hardware that enables movement in one or more directions. For example, the mount 540 may include a gimbal fixedly attached to the structure 550 and rotationally attached to the first section 510. Alternatively, the mount 540 may include a gimbal fixedly attached to the first section 510 and rotationally attached to the structure 550. As another example, a mechanical or robotic arm may be mounted to the structure 550, and either end (structure 550 attachment point or shield mount 540 attachment point) may rotate via a motorized rotary stage. The mechanical or robotic arm may have the ability to hold a load in a variety of static positions. The various configurations allow the rotational movement in three dimensions, described above.

In alternative embodiments, the mount 540 may connect either of the second or third sections 520 or 530 to the structure 550 without departing from the scope of the present teachings. Also, although FIG. 5 shows the mount 540 connecting to the first section 510 from above, it is understood that the mount 540 may connect from below in alternative configurations, without departing from the scope of the present teachings. For example, the structure 550 may be the floor, or a structure sitting on or attached to the floor, to which the mount 540 is fixedly or rotationally attached.

In the depicted embodiment, the control unit 105 and the control IF 144 provide electronic control of the configuration of the radiation shield 542. It is understood, however, that movement of the radiation shield 542 may be fully or partially manual using physical handles or holds (not shown), for example, mounted on one or more of the first, second or third sections 510, 520 and 530, or through direct contact with the first, second or third sections 510, 520, and 530. When controlled electronically through the control unit 105, the configuration of the radiation shield 542 may be performed by sending signals to the motors controlling the first, second or third sections 510, 520, and 530 via the control IF 144. In this case, the sections and/or panels of the radiation shield 542 may be encoded such that the exact configuration of the radiation shield 542 is available to the control unit 105. The encoding provides position coordinates for the radiation shield 542, for example, using sensors and/or translating movement of control motors. Movement of the radiation shield 542 to adjust its configuration may therefore be controlled using buttons on a touch screen or other user interfaces of the control unit 105, such as user interface 112 and/or GUI 118 in FIG. 1, discussed above. Both manual and electronic control of the sections described above may also be applied to the upper and lower panels 511, 512, 521, 522, 531 and 532.

When the radiation shield 542 is encoded, the control unit 105 may be configured to visualize the configuration of the radiation shield on a display interface, such as display 114 in FIG. 1, using a three-dimensional model of the radiation shield 542. The visualization may include information about the shape, position and orientation of the radiation shield 542. If the control unit 105 has a communication channel with the X-ray imaging system 130, the visualization may include the shape, position and orientation of the radiation shield 542 relative to the shape, position and orientation of the C-arm 133. The visualization may additionally show mapping of the radiation patterns, discussed above.

In various embodiments, the control IF 144 may include drive devices, including small motors, such as servo motors or stepper motors, and/or solenoids, configured to provide torque and rolling energy for folding and unfolding the second and third sections 520 and 530, and for vertically moving one or more of the upper and lower panels 511, 512, 521, 522, 531 and 532, respectively. For folding and unfolding operations, the mechanical connections and devices may include motors configured to rotate spools to adjust cables connected to the corners of the second and third sections 520 and 530, for example, as discussed below with reference to FIG. 6. For vertical movement of the panels, the mechanical connections and devices may include motors configured to rotate spools to adjust cables connected to the lower panels 511, 521 and 531 or the upper panels 512, 522 and 532 for sliding the connected panels relative to the other panels, for example, as discussed below with reference to FIGS. 8 and 9. For translational movement of the panels, the mechanical connections and devices may include motors connected to rotate spools to adjust cables at the first section 510 connected to the corners of the second and third sections 520 and 530 for translating the second and third sections relative to the first section 510, for example, as discussed below with reference to FIGS. 7A and 7B. It is understood that any compatible electrical and mechanical connections and devices able to configure the sections and panels of the radiation shield 542 may be incorporated, without departing from the scope of the present teachings.

In the depicted embodiment, the control unit 105 is configured to operate the motors of the control IF 144 to rotate the at least one of the second and third sections 520 and 530 relative to the first section 510 for folding and unfolding the second and third sections to change the width of the coverage area of the radiation shield 542. The control unit 105 is further configured to operate the motors to move one or more of the upper and lower panels 511, 512, 521, 522, 531 and 532 relative to one another to change the height of the coverage area of the radiation shield 542. The control unit 105 is further configured to operate the motors of the control IF 144 to translate at least one of the second and third sections 520 and 530 relative to the first section 510 to change the width of the coverage area of the radiation shield 542.

As discussed above, each of these functions may be performed manually or electronically. In an embodiment, the control unit 105 may include a mechanism (e.g., a switch) that enables the user to change between manual control of the radiation shield 542 and electronic control of the radiation shield 542 by the control unit 105. The manual control enables the user to physically maneuver the first, second and third sections 520 and 530 and/or the upper and lower panels 511, 512, 521, 522, 531 and 532 manually. The electronic control enables the user to operate the control unit 105, through the user interface 112 and/or the GUI 118, to manipulate the first, second and third sections 510, 520 and 530 and/or the upper and lower panels 511, 512, 521, 522, 531 and 532 by operation of motors, solenoids, and/or other electronic control devices. The control unit 105 may also be configured to automatically manipulate the first, second and third sections 510, 520 and 530 and/or the upper and lower panels 511, 512, 521, 522, 531 and 532, as discussed above.

Notably, the configuration shown in FIG. 5 is merely illustrative. It is understood that the configurations of the radiation shield 542 are not limited to these examples, and may vary to provide unique benefits for any particular situation or to meet specific requirements of various implementations, as would be apparent to one skilled in the art. For example, the degrees of folding of one or both of the second and third sections 520 and 530 relative to the first section 510 may vary from zero to about 180 degrees. Likewise, the distance with which one or more of the lower panels 512, 522 and 532 are able to move upwardly relative to the upper panels 511, 521 and 531 respectively may vary from zero to almost 100 percent overlap. Likewise, the degrees of rotation about the y-axis at the mount 540 may vary from zero to ±180 degrees, and the degrees of rotation about one or more of the x-axis and the z-axis at the mount 540 may vary from zero to about ±110 degrees, for example.

FIGS. 6-9 are perspective views of connectors for moveably connecting sections and panels of the configurable radiation screen, according to representative embodiments. FIGS. 6-9 provide examples of the various connectors, and are not limiting.

In particular, FIG. 6 is a perspective view of a connection system movably connecting the first and second sections of the radiation shield for rotational movement, according to a representative embodiment.

Referring to FIG. 6, illustrative connection system 600 includes slot and pin assembly 630 for attaching the first and second sections 510 and 520 for rotational (and translational) movement relative to one another. The connection system 600 further includes a screw actuator 610 positioned on the upper edge of the first section 510, and a protrusion 621 and rollers 425 and 426 positioned on the upper edge of the second section 520. The screw actuator 610 is operable to rotate a worm gear 615, which mechanically interacts with the protrusion 621 on the second section 620. In the depicted configuration, rotation of the worm gear 615 in a first direction causes corresponding rotation of the protrusion 621 around a pin 622, which results in the second section 520 unfolding away from the first section 510. Rotation of the worm gear 615 in an opposite second direction causes corresponding rotation of the protrusion 621 the pin 622, which results in the second section 520 folding toward the first section 510. The rollers 425 and 626 roll along the surface of the second section 520 during a translational movement of the second section 520 relative to the first section 510, discussed below, and impart a force on the second section 520 during the rotation around the pin 622. The screw actuator 610 includes a motor, for example, the operation (e.g., speed and direction of rotation) of which is controlled by electrical signals from the control unit 105. Although the screw actuator 610 may be operated hydraulically or pneumatically, for example, without departing from the scope of the present teachings.

FIG. 7A is a perspective view of a connection system movably connecting the first and second sections of the radiation shield for translational movement, according to a representative embodiment. FIG. 7B is a perspective view of a spool in the connector operable to provide translational movement of the first and second sections of the radiation shield, according to a representative embodiment.

Referring to FIG. 7A, illustrative connection system 700 includes a spool 715 and a motor 718 in a housing 710 connected to the upper edge of the first section 510. The connection system 700 further includes first and second corner connectors 721 and 722 on the left and right corners of the upper edge of the second section 520, and thin first and second steel cables 731 and 732 running between the first and second corner connectors 721 and 722 and the spool 715, respectively. The motor 718 is operable to rotate the spool 715 clockwise and counterclockwise, which in turn complementarily adjusts the lengths of the first and second steel cables in order to translationally move the second section 520 left and right via the first and second corner connectors 721 and 722. The rollers 625 and 626 roll along the surface of the second section 520 during the translational movement.

Referring to FIG. 7B, in the depicted example, clockwise rotation of the spool 715 shortens the first steel cable 731 and lengthens the second steel cable 732, pulling the second section 720 to the right via the first and second corner connectors 721 and 722. Counterclockwise rotation of the spool 715 shortens the second steel cable 732 and lengthens the first steel cable 731, pulling the second section 720 to the left via the first and second corner connectors 721 and 722. The motor 718 may be controlled by electrical signals from the control unit 705, for example. However, in alternative configurations, the spool 715 may be operated hydraulically or pneumatically, for example, without departing from the scope of the present teachings.

FIG. 8 is a perspective view of a connection system movably connecting the upper and lower panels of the third section of the radiation shield for vertical movement, according to a representative embodiment.

Referring to FIG. 8, illustrative connection system 800 includes a spool 815 and a dedicated motor 818 in the housing 710 connected to the upper edge of the first section 510. The connection system 800 further includes first and second corner connectors 821 and 822 on the left and right corners of the upper edge of the lower panel 532 of the third section 530, and thin first and second steel cables 831 and 832 running between the first and second corner connectors 821 and 822 and the spool 815, respectively. The first and second steel cables 831 and 832 are wound in the same direction around the spool 815. The first and second corner connectors 821 and 822 are respectively positioned within rails 841 and 842 attached at the left and right edges of the upper panel 531 of the third section 530. The rails 841 and 842 are configured to guide the vertical movement of the lower panel 532.

The motor 818 is operable to rotate the spool 815 clockwise and counterclockwise, which in turn adjusts the lengths of the first and second steel cables in the same direction in order to vertically move the lower panel 532 down and up relative to the upper panel 531 via the first and second corner connectors 821 and 822. That is, in the depicted example, clockwise rotation of the spool 815 lengthens the first and second steel cables 831 and 832, lowering the lower panel 532 (inside the first and second rails 841 and 842) relative to the upper panel 531 via the first and second corner connectors 821 and 822. Counterclockwise rotation of the spool 815 shortens the first and second steel cables 831 and 832, raising the lower panel 532 relative to the upper panel 531 via the first and second corner connectors 821 and 822. The motor 818 may be controlled by electrical signals from the control unit 105, for example. However, in alternative configurations, the spool 815 may be operated hydraulically or pneumatically, for example, without departing from the scope of the present teachings. Notably, a connection system movably connecting the upper and lower panels 521 and 522 of the second section 520 of the radiation shield 542 for vertical movement would be substantially the same as the connection system 800.

Similarly, FIG. 9 is a perspective view of a connection system movably connecting the upper and lower panels of the first section of the radiation shield for vertical movement, according to a representative embodiment.

Referring to FIG. 9, illustrative connection system 900 includes a spool 915 and a dedicated motor 918 in the housing 710 connected to the upper edge of the first section 510. The connection system 900 further includes first and second corner connectors 921 and 922 on the left and right corners of the upper edge of the lower panel 512 of the first section 510, and thin first and second steel cables 931 and 932 running between the first and second corner connectors 921 and 922 and the spool 915, respectively. The first and second steel cables 931 and 932 are wound in the same direction around the spool 915. The first and second corner connectors 921 and 922 are respectively positioned within rails 941 and 942 attached at the left and right edges of the upper panel 511 of the first section 510. The rails 941 and 942 are configured to guide the vertical movement of the lower panel 512.

The motor 918 is operable to rotate the spool 915 clockwise and counterclockwise, which in turn adjusts the lengths of the first and second steel cables 931 and 932 in the same direction in order to vertically move the lower panel 512 down and up relative to the upper panel 511 via the first and second corner connectors 921 and 922. That is, in the depicted example, clockwise rotation of the spool 915 lengthens the first and second steel cables 931 and 932, lowering the lower panel 531 (inside the first and second rails 941 and 942) relative to the upper panel 511 via the first and second corner connectors 921 and 922. Counterclockwise rotation of the spool 915 shortens the first and second steel cables 931 and 932, raising the lower panel 512 relative to the upper panel 511 via the first and second corner connectors 921 and 922. The motor 918 may be controlled by electrical signals from the control unit 105, for example. However, in alternative configurations, the spool 915 may be operated hydraulically or pneumatically, for example, without departing from the scope of the present teachings.

Although the present specification describes components and functions that may be implemented in particular embodiments with reference to particular standards and protocols, the disclosure is not limited to such standards and protocols. Such standards are periodically superseded by more efficient equivalents having essentially the same functions. Accordingly, replacement standards and protocols having the same or similar functions are considered equivalents thereof.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

1. A system for reducing radiation exposure in a procedure room using a radiation shield, the system comprising:
a controller comprising a processor and memory, the processor configured to:
receive first position data indicating a position of at least one clinician in a procedure room;
receive second position data indicating a position of an imaging source of an imaging device configured to provide image data of a patient in the procedure room;
estimate a radiation pattern of radiation emitted by the imaging source based on the first position data and the second position data; and
predict an optimal position of a radiation shield in the procedure room that minimizes exposure of the at least one clinician to the emitted radiation based on the estimated radiation pattern, the first position data, and the second position data.

2. The system of claim 1, wherein the radiation shield comprises a plurality of interconnected sections, wherein:
at least one section of the plurality of interconnected sections is configured to fold and unfold relative to another section of the plurality of interconnected sections for changing a coverage area of the plurality of interconnected sections, and
at least one section of the plurality of interconnected sections comprises a plurality of panels, wherein at least one panel of the plurality of panels is configured to slide relative to another panel of the plurality of panels for changing the coverage area of the plurality of interconnected sections.

3. The system of claim 2, wherein the prediction of the optimal position of a radiation shield that minimizes exposure of the at least one clinician to the emitted radiation comprises prediction of: (i) a position of the at least one section of the plurality of interconnected sections relative to the another section of the plurality of interconnected sections and (ii) a position of the at least one panel of the plurality of panels relative to the another panel of the plurality of panels.

4. The system of claim 1, wherein the processor is further configured to apply a radiation model configured to estimate the radiation pattern based on the first position data and the second position data.

5. The system of claim 4, wherein the radiation model is further configured to estimate the radiation based on settings of the imaging source, wherein the settings include at least one of dosage, frame rate, exposure time, and collimation of the radiation emitted by the X-ray source.

6. The system of claim 1, wherein the processor is further configured to apply a shield positioning model configured to predict the optimal position of the radiation shield based on the position of the at least one clinician indicated by the first position data, the position of the imaging source indicated by the second position data, and the estimated radiation pattern.

7. The system of claim 6, wherein the shield positioning model is a machine-learning model that comprises at least one of an artificial neural network (ANN) algorithm, a convolutional neural network (CNN) algorithm, and a recurrent neural network (RNN) algorithm.

8. The system of claim 6, further comprising a second processor configured to train the shield positioning model, the second processor configured to:
receive previous first position data indicating positions of the at least one clinician during previous procedures;
receive previous second position data indicating positions of the imaging source during the previous procedures;
receive radiation patterns of the imaging during the previous procedures based on the previous first position data and the previous second position data;
input the previous first position data, the previous second position data, and the estimated radiation patterns to the shield positioning model;
estimate, using the shield positioning model, an optimal configuration of the radiation shield that minimizes exposure of the at least one clinician to radiation from the imaging source; and
adjust parameters of the shield positioning model based on a difference between the estimated optimal configuration and a ground truth optimal configuration of the radiation shield.

9. The system of claim 6, wherein the shield positioning model comprises one or more loss functions configured to predict the optimal position based on at least one of:
minimization of radiation exposure to a clinician of the at least one clinician standing closest to the imaging source;
avoidance of obscuring or inhibiting access to a region of interest; and
protection of an anatomical region of interest from the imaging radiation.

10. The system of claim 1, further comprising:
the radiation shield comprising the radiation shielding material; and
at least one sensor configured to provide at least one of the first position data, the second position data, and position data of one or more other objects in the procedure room.

11. The system of claim 10, further comprising:
at least one motor configured to move at least a portion of the radiation shield,
wherein the processor is further configured to operate the at least one motor to move the radiation shield to the optimal position.

12. The system of claim 10, wherein the at least one sensor comprises an internal encoder configured to receive motion data indicating movement of the motion source during the procedure, wherein the second position data comprises the motion data.

13. The system of claim 1, wherein:
the first position data further indicates a position of a region of interest on the patient, and
the processor is further configured to predict the optimal position of the radiation shield to at least one of (i) prevent the radiation shield to obscure or inhibit access to the region of interest and (ii) protect of the region of interest from the X-ray radiation.

14. The system of claim 1, wherein the first position data further indicates positions of one or more objects in the procedure room, including at least one of the radiation shield and an operating table.

15. A method for reducing radiation exposure in a procedure room using a radiation shield, the method comprising:
determining first position data indicating a position of at least one clinician in a procedure room;
determining second position data indicating a position of an imaging source of an imaging device configured to provide image data of a patient in the procedure room;
estimating a radiation pattern of radiation emitted by the imaging source based on the first position data and the second position data; and
predicting an optimal position of a radiation shield in the procedure room that minimizes exposure of the at least one clinician to the emitted radiation based on the estimated radiation pattern, the first position data, and the second position data.

16. The method of claim 15, wherein the radiation shield comprises a plurality of interconnected sections, wherein:
at least one section of the plurality of interconnected sections is configured to fold and unfold relative to another section of the plurality of interconnected sections for changing a coverage area of the plurality of interconnected sections, and
at least one section of the plurality of interconnected sections comprises a plurality of panels, wherein at least one panel of the plurality of panels is configured to slide relative to another panel of the plurality of panels for changing the coverage area of the plurality of interconnected sections; and
the predicting of the optimal position of a radiation shield that minimizes exposure of the at least one clinician to the emitted radiation comprises prediction of: (i) a position of the at least one section of the plurality of interconnected sections relative to the another section of the plurality of interconnected sections and (ii) a position of the at least one panel of the plurality of panels relative to the another panel of the plurality of panels.

17. The method of claim 15, wherein the predicting of the optimal position of a radiation shield comprises apply a shield positioning model comprising a machine-learning model trained to predict the optimal position of the radiation shield based on the position of the at least one clinician indicated by the first position data, the position of the imaging source indicated by the second position data, and the estimated radiation pattern.

18. A non-transitory computer readable medium having stored a computer program comprising, which, when executed by a processor, cause the processor to:
determine first position data indicating a position of at least one clinician in a procedure room;
determine second position data indicating a position of an imaging source of an imaging device configured to provide image data of a patient in the procedure room;
estimate a radiation pattern of radiation emitted by the imaging source based on the first position data and the second position data; and
predict an optimal position of a radiation shield in the procedure room that minimizes exposure of the at least one clinician to the emitted radiation based on the estimated radiation pattern, the first position data, and the second position data.

19. The non-transitory computer readable medium of claim 18, wherein the radiation shield comprises a plurality of interconnected sections, wherein:
at least one section of the plurality of interconnected sections is configured to fold and unfold relative to another section of the plurality of interconnected sections for changing a coverage area of the plurality of interconnected sections, and
at least one section of the plurality of interconnected sections comprises a plurality of panels, wherein at least one panel of the plurality of panels is configured to slide relative to another panel of the plurality of panels for changing the coverage area of the plurality of interconnected sections; and
the prediction of the optimal position of a radiation shield that minimizes exposure of the at least one clinician to the emitted radiation comprises prediction of: (i) a position of the at least one section of the plurality of interconnected sections relative to the another section of the plurality of interconnected sections and (ii) a position of the at least one panel of the plurality of panels relative to the another panel of the plurality of panels.

20. The non-transitory computer readable medium of claim 18, wherein to predict the optimal position of a radiation shield, the instruction, when executed by the processor, further cause the processor to:
apply a shield positioning model comprising a machine-learning model trained to predict the optimal position of the radiation shield based on the position of the at least one clinician indicated by the first position data, the position of the imaging source indicated by the second position data, and the estimated radiation pattern.
